(19) United States
(12) Patent Application Publication
(10) Pub. No.: US 2018/0070801 A1
(43) Pub. Date: Mar. 15, 2018

(54) APPARATUS FOR VISUAL EXAMINATION WITHIN CAVITIES

(71) Applicant: TYTO CARE LTD., Netanya (IL)

(72) Inventors: Mordechai TEICHER, Hod Hasharon (IL); David GlLAD-GILOR, Even Yehuda (IL); Eyal BYCHKOV, Hod Hasharon (IL)

(21) Appl. No.: 15/563,474

(22) PCT Filed: Mar. 24, 2016
(86) PCT No.: PCT/IL2016/050323

§ 371 Date: Sep. 29, 2017

Related U.S. Application Data
(60) Provisional application No. 62/140,545, filed on Mar. 31, 2015.

(51) Int. Cl. A61B 1/00; A61B 1/247; A61B 1/04

(52) U.S. Cl. CPC A61B 1/00147; A61B 1/00016; A61B 1/06; A61B 1/247; A61B 1/04; A61B 1/0014

(57) ABSTRACT

An apparatus for inspection within cavities includes a thimble having a camera and a receptacle for engaging with a distal end of a selected finger of a user, so that the camera can be positioned and oriented by the user within the cavity. The apparatus is useful, for example, for throat examination of babies or children.

### FIELD

The present invention relates to visual examination within cavities, and in particular to a medical device for visual examination of a patient's mouth and throat.

### BACKGROUND

Doctors routinely look into a patient's mouth to diagnose a sore throat. To get a good look at the throat and tonsils, the patient is required to keep his or her mouth wide open while the doctor uses a tongue depressor and a flashlight. Such a procedure is often inconvenient to many patients, and becomes challenging with babies and children, as well as with people who tend to have a pharyngeal (or “gag”) reflex when a tongue depressor is used.

Throat exams can be made also remotely over the Internet, by patients or family members taking and sending an image of the throat to a doctor, or sharing with the doctor a live view of the throat. However, capturing images of the throat is challenging for the reasons recited above, especially when users are untrained and inexperienced.

Certain embodiments of the present invention reduce the difficulty of throat examinations, both at the clinic and at home. Other embodiments are useful for examination within other body cavities of humans or animals, as well as for visual inspection within other hard-to-reach cavities.

### Definitions

A “patient” is herein a person whose throat is to be examined.

A “user” is herein a person performing a throat examination, and can be the patient (for self examination), a doctor, a nurse, a family member, or any other person assisting a patient. A user is also any person visually examining or inspecting features within any other cavity where embodiments of the present invention can be useful, such as inside other body cavities of humans or animals, and some other cavities that are hard to reach with conventional cameras.

### SUMMARY

For making the description below concrete and tangible, the present disclosure focuses on examining the mouth cavity of a human, particularly for throat examination. It will be appreciated, however, that embodiments of the present invention are applicable and useful for examining other body cavities of humans or animals, as well as for inspecting some other hard-to-reach cavities. Accordingly, illustrations in the context of mouth and throat examination are non-limiting; and the terms “mouth” and “throat” hereinbelow have corresponding counterparts in a large variety of cavities and inspection targets.

The term “thimble” herein denotes a cap or cover that may be worn on the end of a finger or a thumb. Various embodiments of the present invention include a thimble for easily mounting small devices on the end of a finger.

Embodiments of the present invention provide a thimble that includes a camera, which is securely attached to a user's finger, such as the index or the thumb. The user then inserts the finger into the patient's mouth, so that the camera captures a live view of the throat and tonsils, and transmits the picture to a nearby computer that displays the viewed image on the screen, thus providing feedback that helps the user in positioning and orienting the camera by the finger until the view reaches sufficient detail and clarity, possibly also toward taking one or more still images and/or a video clip, if the examination is made at home by a user who is the patient or a family member, the live view may be shared online with a doctor, who may provide further instructions for guiding the user in positioning and orienting the camera by his finger.

According to preferred embodiments there is provided an apparatus for inspection within cavities, including a thimble that has a camera for capturing a view within a cavity, the camera having optics with an optical axis. The thimble further includes a receptacle for engaging with a distal end of a selected finger of a user for positioning and orienting the thimble within the cavity by the user, and the receptacle is pre-aligned with the camera so that the optical axis is substantially parallel to the distal end of the selected finger. The apparatus also includes a finger attachment for securing the thimble to the selected finger beyond the distal end, and a short-range connection for sending the view captured by the camera to a computer. The finger attachment may form part of a disposable hygienic cover that includes a protective sheath for protecting the finger and a clear tip so that the camera can capture the view through the clear tip. The hygienic cover may form part of a disposable glove. The camera may have an illuminator for illuminating a part of the cavity, and the cavity may be within a mouth of a patient while the view is of the patient's throat or teeth. The apparatus may further include a cuff for securing the apparatus to a wrist of the user and host a battery, a processor and/or a short-range connection unit.

Also provided is a method of inspection of cavities by a user, the method including the steps of: (i) attaching a thimble, that includes a camera, to a distal end of a selected finger of the user, so that an optical axis of the camera is substantially parallel to the distal end of the selected finger; (ii) inserting the selected finger into a cavity; (iii) positioning the thimble within the cavity by the selected finger; (iv) capturing by the camera a view within the cavity; (v) displaying the view on a computer screen; and repeating the positioning, capturing and displaying steps while moving the thimble within the cavity by the finger. The method may be used for a cavity within a mouth of a patient, for capturing a view of the patient's throat or teeth.

Further provided is a disposable hygienic cover for attaching a thimble having a camera to a finger of a user, the disposable hygienic cover including a protective sheath for protecting the finger and for securing the thimble to the distal end of the finger, and a clear tip at the end of the protective sheath to allow the camera to capture views through the clear tip. The disposable hygienic cover may form part of a disposable glove.

Using the thimble according to embodiments of the present invention offers important advantages:
High level of user control ver the camera position and orientation within the mouth.
Intuitive, effective operation by untrained users—ideal for home and remote medicine.
Safe, friendly and intuitive operation when used by a parent to examine the throat of a baby or a child.
Friendly and effective method for self examination by patients of all ages, including children who may think of the examination as a computer game.
Self operation by a patient overcomes or reduces the gag reflex when depressing the tongue is required for an adequate view.

### BRIEF DESCRIPTION OF SEVERAL VIEWS OF THE DRAWINGS

The present invention will be understood and appreciated more fully from the following detailed description, taken in conjunction with the drawings in which:

FIG. 1 is a simplified block diagram describing a system according to a preferred embodiment of the present invention.

FIG. 2 is a schematic illustration of a thimble according to a preferred embodiment of the present invention.

FIGS. 3A-3B are schematic illustrations of apparatuses according to preferred embodiments of the present invention.

FIGS. 4A-4C are schematic illustrations of exemplary apparatuses that include hygienic covers.

FIGS. 5A-5B are schematic illustrations of standalone thimbles.

FIGS. 6-8 are schematic illustrations of exemplary use scenarios.

FIG. 9 is a simplified flowchart describing the operation of an apparatus according to a preferred embodiment of the present invention.

### DETAILED DESCRIPTION

### The System

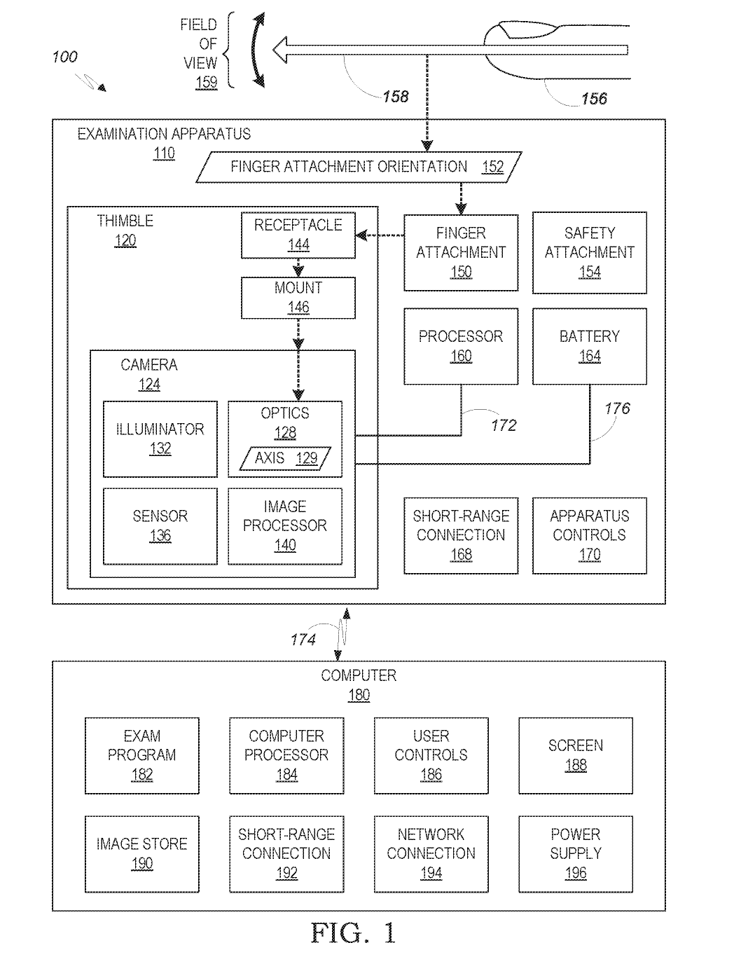

Reference is made to FIG. 1, which is a schematic block diagram of an examination system 100 according to an embodiment of the present invention. Examination system 100 include an examination apparatus 110 that communicates via communication link 174 with a computer 180.

Examination apparatus 110 includes a thimble 120, to be worn on the distal end of a finger of a user order to inspect the throat or teeth of a patient. Thimble 120 includes a camera 124 for taking live, still and/or video images of the throat or teeth, and a receptacle 144 to engage with distal end 156 of a user's finger, such as the index finger or the thumb, via a finger attachment 150.

Finger distal end 156 points in finger axial direction 158. When distal end 156 is gripped by finger attachment 150, finger attachment orientation 152 aligns with finger axial direction 158. Finger attachment 150 is pre-aligned with receptacle 144, which is pre-aligned with a mount 146 for camera 124, which includes camera optics 128 having an optical axis 129. These pre-alignments are such that optical axis 129 is substantially parallel to finger attachment orientation 152. Therefore, when the user wears thimble 120, finger attachment 150 grips distal end 156 so that optical axis 129 is substantially parallel to the finger distal end 156, i.e. to finger axial direction 158.

Thus, when the user points finger distal end 156 in the direction of a desired field of view 159, optical axis 129 of camera optics 128 will align in the proper direction to conveniently capture an image of the features within field of view 159 at which the user is pointing.

In addition to camera optics 128, which is suited to capture an image of the throat and tonsils or the teeth from within the patient's mouth, camera 124 also preferably includes an illuminator 132 to illuminate the throat and tonsils or the teeth, an image sensor 136 to digitize the image captured by optics 128, and an image processor 140 to convert the output of sensor 136 into a usable format of a still image or video stream.

Finger attachment 150 includes physical elements to attach examination apparatus 110 to the finger beyond the distal end, for further securing thimble 120 to the user's finger, as well as optionally for routing wires used in data link 172 and power connection 176 along the finger. Safety attachment 154 is optionally included for securing thimble 120 to the wrist or another part of the user's hand, to further eliminate the risk of thimble 120 being lost within the patient's mouth. Processor 160 controls the operation of all active units of examination apparatus 110, and cooperates with apparatus short-range connection 168, such as a Bluetooth transceiver or a wired connection, to send still images and/or video streams to computer 180 and receive from computer 180 operational commands, via communication link 174, such as a Bluetooth link or physical wire. It will be noted that, in some embodiments, image processor 140 and processor 160 may be merged into a single physical processor. Battery 164, such as a rechargeable or a replaceable battery, energizes all active components of examination apparatus 110. Data link 172 allows processor 160 to control the operation of camera 124 and receive image data therefrom, while power connection 176 energizes camera 124 from battery 164. In some embodiments, data link 172 and power connection 176 may be embodies as a multi-wire data/power connection, such as a USB connection. In some embodiments, apparatus controls 170 are included for allowing the user to control on/off and camera settings from examination apparatus 110, while in other embodiments some or all such control actions are made via user controls 186 of computer 180.

Computer 180 is preferably a standard computing device, such as a desktop, laptop or tablet computer or a mobile phone, that is positioned next to the user of examination apparatus 110 during the throat or teeth exam operation, and is programmed to cooperate with examination apparatus 110. Computer 180 may also be a dedicated device that is supplied with examination apparatus 110. Computer 180 includes computer processor 184 that executes an exam program that is stored in exam program nonvolatile storage device 182, to receive via computer short-range connection 192 and communication link 174, display on screen 188, and possibly store in image store 190 and retransmit via network connection 194, the live view of the throat or teeth captured by camera 124 of thimble 120. User controls 186, such as a keyboard, a mouse or a touchscreen, allow the user to control the operation of computer 180, and possibly also of examination apparatus 110. Screen 188 that forms part of computer 180 or is connected to it, is positioned for convenient watching by the user during the exam process. Image store 190 is a nonvolatile storage device, such as a hard disk or flash memory, for storing still images and/or video clips captured by camera 124. Computer short-range connection 192, such as Bluetooth connection or a wire connection, uses communication link 174 to connect with examination apparatus 110 via short-range connection 168. Network connection 194, such as a wired or Wi-Fi Internet connection or a cellular connection, serves to optionally communicate with a remote doctor for sending images and/or receiving live instructions. Power supply 196 is a conventional unit for energizing the components of computer 180.

### The Thimble

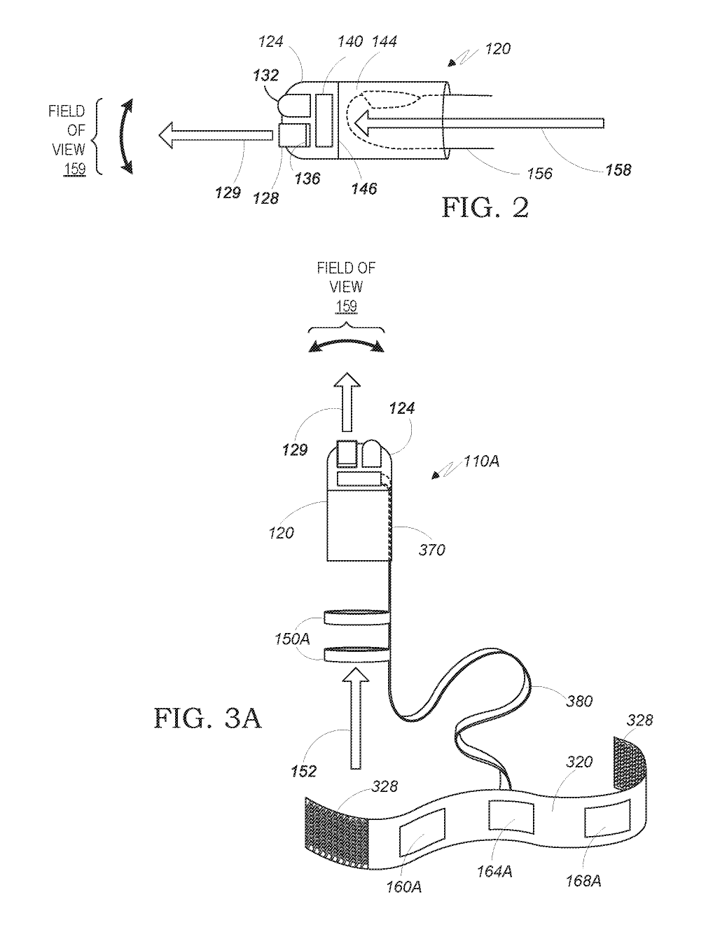

FIG. 2 is a schematic illustration of an example of thimble 120 of FIG. 1. Thus, camera 124 is positioned at the tip of thimble 120, so that its illuminator 132 and optics 128 can effectively capture throat or teeth images during the exam process. Receptacle 144 firmly receives and engages with user finger distal end 156, so that the user can effectively and instinctively position and orient camera 124 during the exam process by simply pointing the finger wearing the apparatus toward the features to be imaged. Several inserts of different sizes and cross sections (not shown in FIG. 2) may be selectively used for optimal adaptation of receptacle 144 to different fingers (e.g. index or thumb) and individual finger sizes.

### The Apparatus

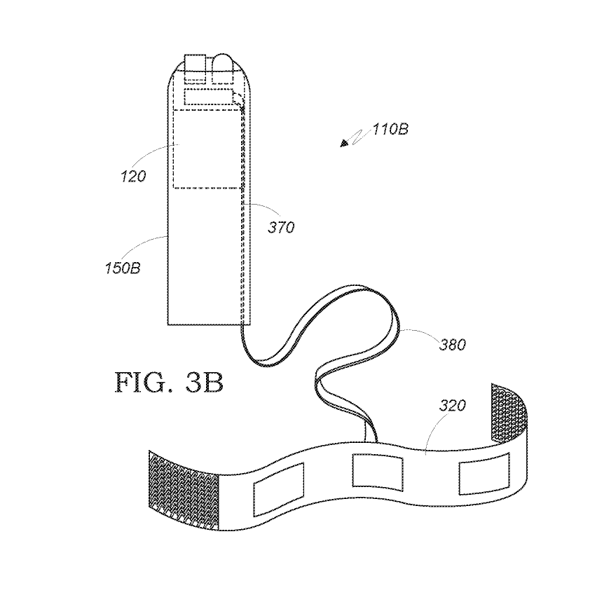

FIG. 3A schematically illustrates examination apparatus 110A, that includes thimble 120 and cuff 320 connected by band 380. Thimble 120 is as depicted with reference to FIG. 2 above, with the addition of connection 370, that includes thin conductors for supplying power from and exchanging data with cuff 320. Connection 370, such as a USB connection, continues to and is embedded within band 380 to provide the functions of data link 172 and power connection 176 recited above with reference to FIG. 1. Cuff 320 includes fasteners 328, such as Velcro® fasteners, for convenient attachment to the user's wrist. Cuff 320 also accommodates processor 160A, battery 164A and short-range connection 168A, to provide the functions of processor 160, battery 164 and short-range connection 168, respectively, recited above with reference to FIG. 1. Band 380 serves both for accommodating connection 370 for power supply and data exchange between cuff 320 and thimble 120, and also for safety, by further securing thimble 120 against falling into the throat and being swallowed by the patient (see also safety attachment 154 of FIG. 1). The two rings that form finger attachment 150A serve to keep a portion of band 380 in place along the operating finger, and also secure thimble 120 to the user's finger beyond the distal end 156 for functionality and safety. Finger attachment orientation 152 is substantially parallel to optical axis 129, so that when the finger is inserted into thimble 120, the user may conveniently point the finger in the direction of the desired field of view 159 for camera 124 to capture an image of the features therein.

FIG. 3B schematically illustrates examination apparatus 110B, that is similar to examination apparatus 110A of FIG. 3A, except that the two rings of finger attachment 150A are replaced by elastic sleeve 150B, such as a latex sleeve. In this embodiment, elastic sleeve 150B maintains optical axis 129 substantially parallel to the axial direction of the distal end of the finger, so that the user can image desired features by conveniently pointing the distal end of the finger at those features.

### Hygienic Cover

For hygienic purpose, the thimble described with reference to FIGS. 3A-3B is preferably washable, which may be adequate for occasional use by a single patient or within a family. However, by personal preference, or when used for multiple unrelated patients, such as in a clinic, a disposable hygienic cover may be advantageous.

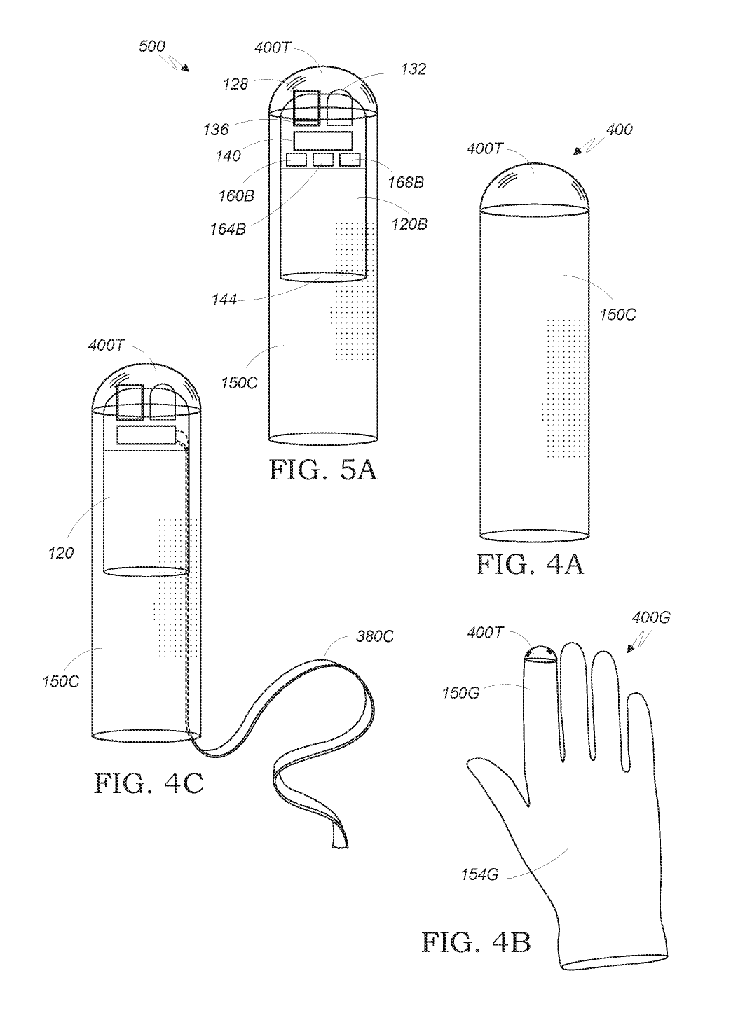

FIG. 4A schematically illustrates a disposable hygienic cover 400, that includes transparent tip 400T, made of a clear material, and protective sheath 150C, such as a latex sheath, that serves both a hygienic purpose and as finger attachment 150 of FIG. 1.

FIG. 4B further extends the concept of disposable hygienic cover 400 of FIG. 4A, into disposable glove 400G, that is similar to latex gloves used by medical staffs, except that one finger sheath, for example the index sheath, is adapted to receive thimble 120. Thus, protective sheath 150G is longer and wider than the corresponding finger sheath in a conventional glove, and has a transparent tip 400T at its end. It will be noted that protective sheath 150G also provides the function of finger attachment 150 of FIG. 1, while the palm portion 154G is also serving as safety attachment 154 of FIG. 1.

FIG. 4C schematically illustrates a thimble 120 inserted within disposable hygienic cover 400 of FIG. 4A, where band 380C includes wires that connect thimble 120 directly to a computer 180, such as a mobile phone (not shown), thereby serving as both short-range connection 168 and communication link 174 of FIG. 1.

### Standalone Thimble

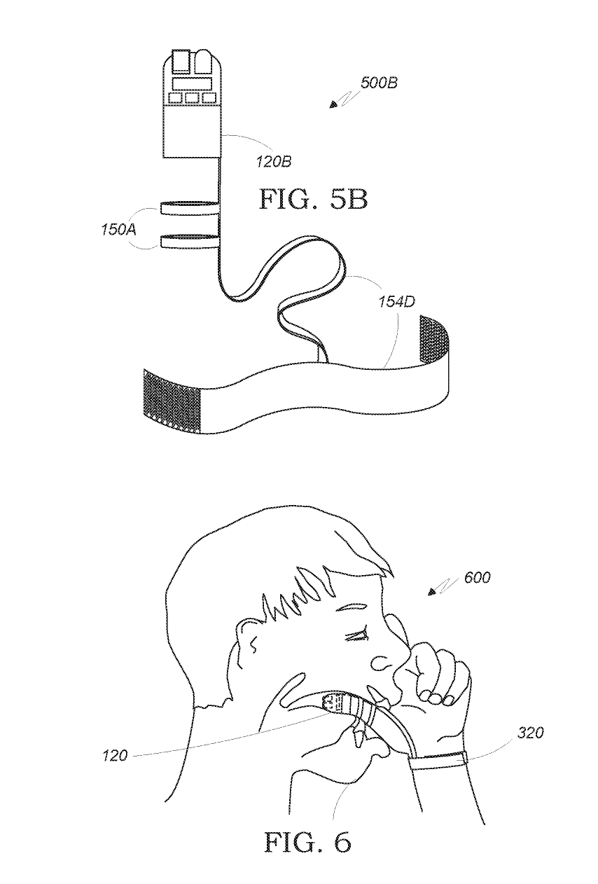

FIG. 5A schematically illustrates an examination apparatus 500, where all functional units of examination apparatus 110 of FIG. 1 are included within a standalone thimble 120B. Thus, standalone thimble 120B now includes optics 128, image sensor 138, illuminator 132 and image processor 140, as well as processor 160B, battery 164B and short-range connection 168B, and, optionally, also apparatus controls 170 of FIG. 1 (not shown). Disposable hygienic cover 400 of FIG. 4A, represented in FIG. 5A by protective sheath 150C and transparent tip 400T, serves both for hygienic purpose and as finger attachment 150 of FIG. 1.

FIG. 5B schematically illustrate an alternative embodiment 500B to the embodiment of FIG. 5A, having no disposable hygienic cover. The two rings of finger attachment 150A serve as finger attachment 150A of FIG. 1 to secure standalone thimble 120B to the user's finger, while optional dumb hand and dumb cuff 154D serve as safety attachment 154 of FIG. 1 for extra safety.

### Exemplary Use Scenarios

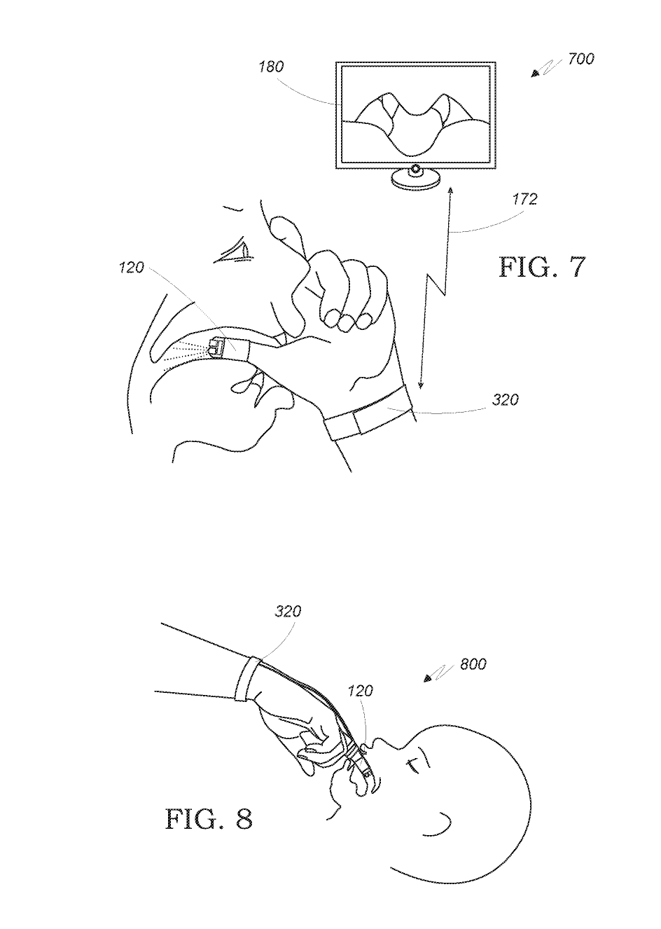

FIG. 6 schematically illustrates use scenario 600, where a user uses his thumb to push thimble 120 so that its camera can view the throat or teeth and cuff 320 is used for sending the captured image to be displayed on a computer screen. It will be noted that the present scenario is of self exam, i.e. the user and the patient are the same person.

FIG. 7 schematically illustrates use scenario 700, that extends the use scenario 600 of FIG. 6 by showing also computer 180 that receives a live view of the throat or teeth from cuff 320 via data link 172. The user watches the screen of the computer so that be can position and direct the thimble's camera with his thumb, until a satisfactory image is displayed on the screen.

FIG. 8 schematically illustrates a use scenario 800, where a mother inserts a thimble 120, secured by cuff 320, into her baby's mouth, for throat examination. It will be noted that in the present scenario, the user and the patient are different persons, as will also be the situation when an examination apparatus 110 is used by medical personnel to perform throat exams at the or at the patient's home.

### Operation

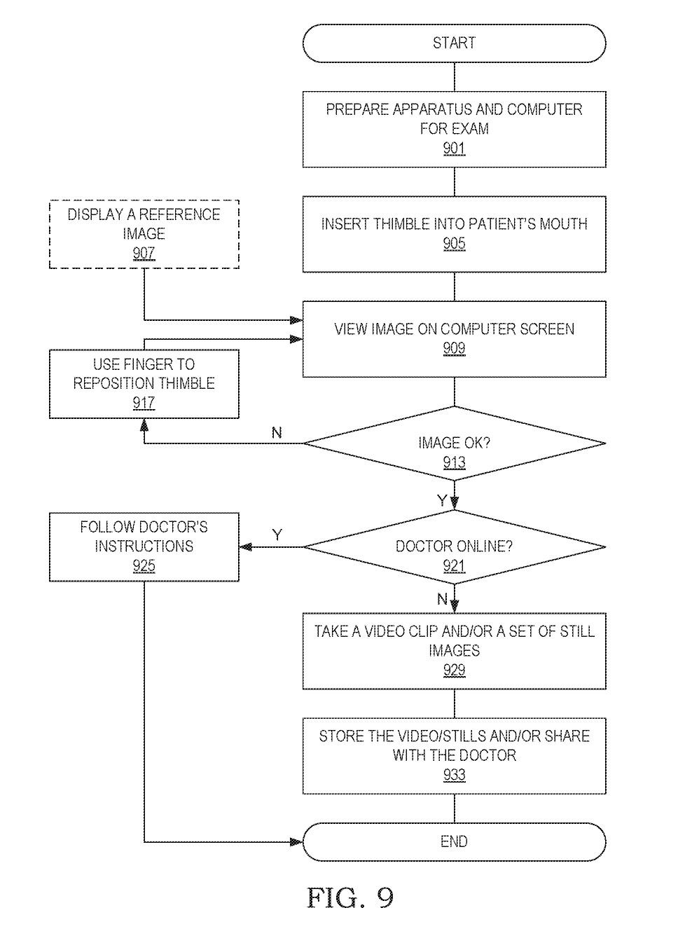

FIG. 9 is a schematic flowchart of an exemplary operation of an apparatus described above. In step 901, the user, who is either the patient or another person, prepares for examination by wearing the thimble on a selected finger and optionally fastening the cuff (if included) around the wrist, and then using controls on the apparatus (thimble or cuff) and/or the computer, to turn on the apparatus and the computer, and loading the exam program to execute on the computer's processor. In step 905 the user uses the selected finger to insert the thimble into the patient's mouth, and in step 909 the user views the image currently captured by the thimble's camera, on the computer's screen. Optionally, step 907 displays on the screen also a reference image, that is either a general reference image of a throat and tonsils or teeth, as a guidance for an unexperienced user, or a previous throat or teeth image of the patient, for comparison by a doctor or by an experienced user in order to detect changes in the throat or teeth condition.

If in step 913 the user finds the image coverage or quality unsatisfactory, then is step 917 the user uses his selected finger to reposition and/or reorient the thimble for improving coverage or quality of the image viewed on the computer's screen. When the user finds, in step 913, the image satisfactory, then step 921 checks the exam scenario: in a first exam scenario, steps 901-913 are carried out by the patient or another person assisting the patient, while a remotely-located doctor watches the live image sent to the doctor's computer by computer 180 via the Internet or another network, and then, in step 925, the user follows the doctor's instructions for further positioning and orienting the thimble's camera using his selected finger. If there is no doctor present online, then step 921 diverts to step 929, where the thimble's camera takes one or more throat or teeth images and/or a video clip and the apparatus sends them to the computer, and in step 933, the taken still images and/or video clip are stored in the image store of the computer and/or are uploaded or emailed to the doctor's server or computer.

The use scenario of throat or teeth examination through the specification and drawings emphasizes a preferred embodiment and serves to provide clear and concrete depictions of structure, operation and usefulness. However, the teachings above are useful and extendable also for the examination and inspection of other body cavities of humans, for body cavities of animals, and for some other hard-to-reach cavities in non-medical scenarios.

While the invention has been described with respect to a limited number of embodiments, it will be appreciated by persons skilled in the art that the present invention is not limited by what has been particularly shown and described herein. Rather the scope of the present invention includes both combinations and sub-combinations of the various features described herein, as well as variations and modifications as would be apparent to a person skilled in the art.

What is claimed is:

1. An apparatus for inspection within cavities, the apparatus comprising:
a thimble that includes:
a camera for capturing a view within a cavity, the camera having optics with an optical axis, and
a receptacle for engaging with a distal end of a selected finger of a user for positioning and orienting the thimble within the cavity by the user, wherein the receptacle is pre-aligned with the camera so that the optical axis is substantially parallel to the distal end of the selected finger; and
a short-range connection for sending the view captured by the camera to a computer.

2. The apparatus of claim 1, further comprising a finger attachment for securing the thimble to the selected finger beyond the distal end.

3. The apparatus of claim 2, wherein the finger attachment forms part of a disposable hygienic cover that includes:
a protective sheath for protecting the finger; and
a clear tip so that the camera can capture the view through the clear tip.

4. The apparatus of claim 3, wherein the hygienic cover forms part of a disposable glove.

5. The apparatus of claim 1, wherein the camera includes an illuminator for illuminating a part of the cavity.

6. The apparatus of claim 1, wherein the cavity is within a mouth of a patient, and the view is of the patient's throat or teeth.

7. The apparatus of any of claims 1-4, further comprising a cuff for securing the apparatus to a wrist of the user.

8. The apparatus of claim 7, wherein the cuff includes at least one of: a battery, a processor or a short-range connection unit.

9. A method of inspection of cavities by a user, the method comprising:
attaching a thimble, that includes a camera, to a distal end of a selected finger of the user, so that an optical axis of the camera is substantially parallel to the distal end of the selected finger;
inserting the selected finger into a cavity;
positioning the thimble within the cavity by the selected finger;
capturing by the camera a view within the cavity;
displaying the view on a computer screen; and
repeating the positioning, capturing and displaying steps while moving the thimble within the cavity by the finger.

10. The method of claim 9, wherein the cavity is within a mouth of a patient, and the view is of the patient's throat or teeth.

11. A disposable hygienic cover for attaching a thimble having a camera to a finger of a user, the disposable hygienic cover comprising:
a protective sheath for protecting the finger and for securing the thimble to the distal end of the finger; and
a clear tip at the end of the protective sheath to allow the camera to capture views through the clear tip.

12. The disposable hygienic cover of claim 11, further comprising four finger covers and a palm cover, so that the disposable hygienic cover is in a form of a disposable glove.
